(19) United States
(12) Patent Application Publication
(10) Pub. No.: US 2021/0145187 A1
(43) Pub. Date: May 20, 2021

(54) ARM SUPPORT FOR CARRYING A BABY

(71) Applicant: Felipe de Barros LEONETTI, Pinhals (BR)

(72) Inventor: Felipe de Barros LEONETTI, Pinhals (BR)

(21) Appl. No.: 16/623,213

(22) PCT Filed: May 27, 2018
(86) PCT No.: PCT/BR2018/050174

§ 371 Date: Dec. 16, 2019

(30) Foreign Application Priority Data
Jun. 19, 2017 (BR) BR202017013077-6

(51) Int. Cl. A47D 13/02; A45F 3/14

(52) U.S. Cl. CPC A47D 13/025; A45F 2003/142; A45F 3/14

(57) ABSTRACT

The arm support for carrying a baby is a lightweight, versatile accessory aimed at making an adult's task of carrying a baby easier. The arm support for carrying a baby is secured to the adult's arm and forms a seat for the baby. The baby is placed in a very comfortable position, with a great deal of contact with the adult's body and with enough ventilation. For the adult, the baby's position affords a sensation of great security, since the adult's forearm is in a position in which it is always encircling the baby. The accessory offers a great advantage over existing models on the market in terms of ease of dressing and accommodating the baby. It also offers the flexibility for the baby to be moved and provides a higher level of safety.

An arm support for carrying a baby (1) is a lightweight, versatile accessory aimed at making an adult's task of carrying a baby easier. The arm support for carrying a baby is secured to the adult's arm and forms a seat (10) to seat the baby. The accessory offers a great advantage over existing models on the market in terms of ease of dressing and accommodating the baby. It also offers the flexibility for the baby to be moved and provides a higher level of safety.

The arm support for carrying a baby (1) is comprised of the body (10), the elbow band (20) and the wrist band (30). In the body (10), there is the body connector (11). In the elbow band (20) there is the elbow connector (21) and the complementary elbow connector (22). In the wrist band (30) there is the wrist connector (31), the complementary wrist connector (32) and the complementary body connector (12). At one of the ends, the body (10) is attached to the elbow band (20) and at the other end it is attached to the wrist band (30). The body (10) is detachable from the wrist band (30). It is not relevant for the present invention to determine the type of material to be employed in the accessory fabric; the important thing is that the chosen material is strong enough to withstand the load generated by the baby's weight. It is also not relevant to determine the types of connectors, as long as they withstand the load thereon. Further, the material and the sewing system of the components are not relevant, as long as they withstand the load.

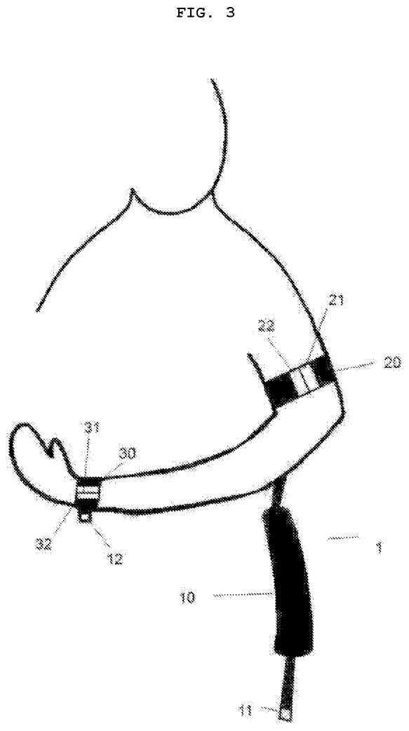

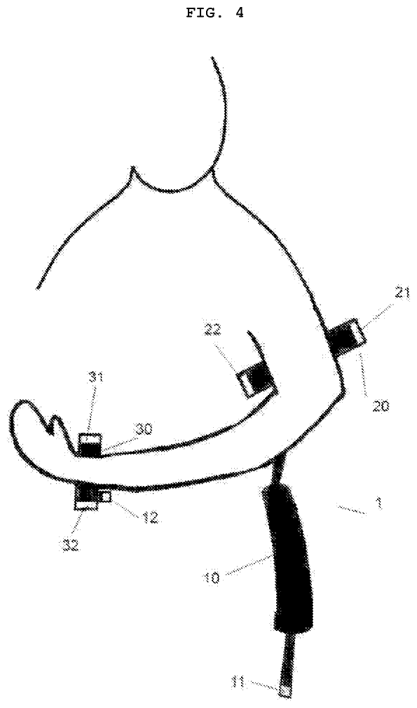

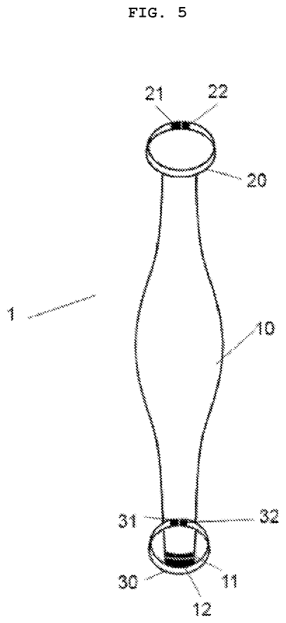

The process of dressing the arm support for carrying a baby (1) involves wrapping the elbow band (20) in the adult's arm by connecting the elbow connector (21) to the complementary elbow connector (22) and wrapping the wrist band (30) to the pulse of the same arm by connecting the wrist connector (31) to the complementary wrist connector (32), as shown in FIG. 3.

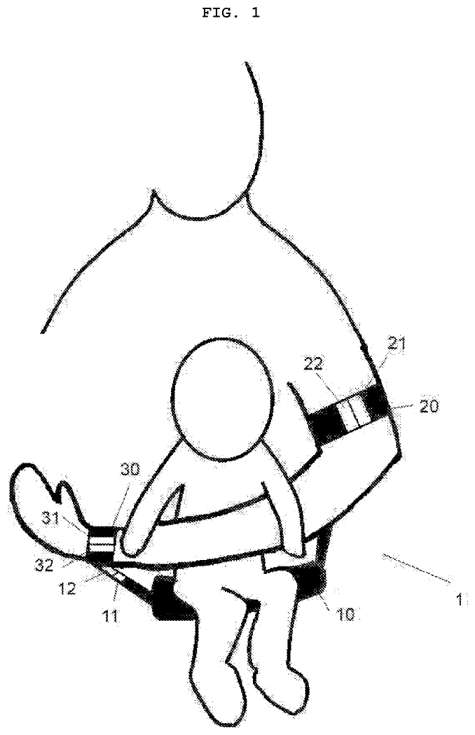

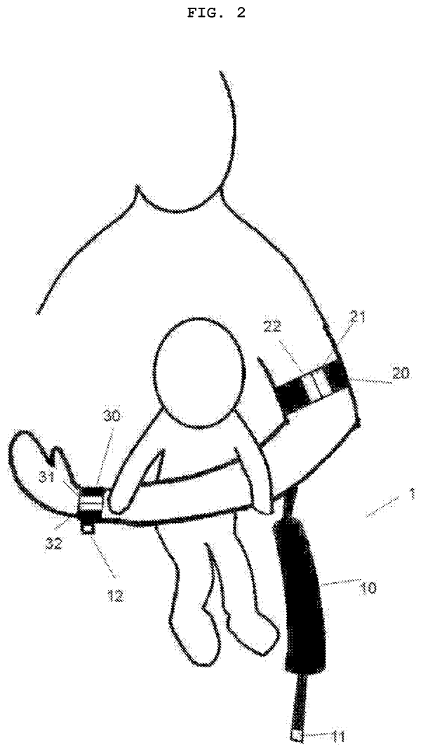

When the adult is wearing the arm support for carrying a baby (1), the baby can be accommodated in the accessory. The body connector (11) must be disconnected from the complementary body connector (12), as shown in FIG. 3. The baby is taken by the abdomen with the arm wearing the accessory, bringing the baby's back to the belly of the adult, as shown in FIG. 2. Then the hand of the free arm is passed under the legs of the baby to pick up the end of the body (10) where the body connector (11) is located and bring it in the direction of the wrist band (30), connecting the body connector (11) to the complementary body connector (12), so that the body (10) touches the buttock of the baby, as shown in FIG. 1. Then the pressure the forearm exercises over the abdomen of the baby is released so that the weight of the baby is gradually transferred to the arm support for carrying a baby (1). The adult's forearm is in front of the baby's chest, preventing the baby from falling forward.

The current state of the art was determined through research contracted with the National Institute of Industrial Property (INPI). The research contemplated the national patent database and international databases. The research has found documents that define the general state of the art, namely: patent references WO2017057895, U.S. Pat. No. 8,523,028, JP4399644B and US2008047987.

Upon analyzing the documents, it is concluded that the current state of the art presents imperfections in the proposed solution of making it easier for adults to carry babies since they present one or more of the following problems:

High complexity to dress the accessory;

Difficulty placing and/or removing the baby from the accessory;

Low ventilation;

It is not indicated for the position where the baby is facing forward (baby's gaze in the same direction as the adult's gaze), with the baby's back against the adult's chest—a position that is very desirable for babies over 3 months old, when curiosity about what happens around them increases and the neck musculature already allow the baby to support and move the head.

The arm support for carrying a baby provides an appropriate solution to remedy the imperfections as listed above, the advantage of the proposed model being obvious regarding the ease of wearing the accessory, the ease of placing in/removing the baby of the accessory, the safety level and the versatility to move the baby.

The procedures for dressing the accessory and placing/removing the baby are described in paragraphs 3 and 4 of this document. The adult easily performs the procedures without needing any help from a third party.

The advantage of the model with respect to the versatility to move the baby refers to the possibility of the adult to easily move the baby to the side of his body (of the adult himself), since the accessory is not fixed to the trunk of the adult. This quality allows the front of the adult body to be released in case it is necessary to handle an object with the free arm hand.

As for safety, the proposed model offers an advantage because the adult's forearm is permanently positioned in front of the baby's chest in a position that prevents the baby from falling forward. The position of the forearm also allows you to use it to defend the baby from any object that may come towards him/her, or to divert the baby from the way of the object by means of a rapid movement of the arm, without risk of twists in the body of the baby that will remain stable due to the compression of the forearm toward the adult's belly.

1. An arm support for carrying a baby comprising a support provided by a band that is secured close to the adult's elbow and another band is secured to the wrist, and a body serving as a seat for the baby, so that the center of mass of the baby is positioned at a height lower than the forearm surrounding the baby.
